(19) United States
(12) Patent Application Publication
(10) Pub. No.: US 2017/0327455 A1
(43) Pub. Date: Nov. 16, 2017

(54) Omefibrates for Treating Dyslipidemia and Cardiovascular Disease

(71) Applicant: Jiva Pharma, Inc., Ann Arbor, MI (US)

(72) Inventor: Om P. Goel, Ann Arbor, MI (US)

(73) Assignee: Jiva Pharma, Inc., Ann Arbor, MI (US)

(21) Appl. No.: 15/663,775

(22) Filed: Jul. 30, 2017

Related U.S. Application Data
(62) Division of application No. 14/721,258, filed on May 26, 2015.
(60) Provisional application No. 62/003,655, filed on May 28, 2014.

(51) Int. Cl. C07C 69/587; C07C 279/26; C07C 57/03

(52) U.S. Cl. CPC C07C 69/587; C07C 57/03; C07C 279/26

(57) ABSTRACT

The present invention relates to the fibric acid derivatives of omega-3 fatty acids and their use in treating Type2 diabetes, obesity, hypertriglyceridemia, cardiovascular diseases, metabolic syndrome, cancer, Alzheimer's disease; and their use for modulating activity of peroxisome proliferator-activated receptors (PPARs).

### FIELD OF THE INVENTION

The present invention relates to novel Omefibrates that are fibric acid type derivatives of omega-3 polyunsaturated acids (fatty acids) used for treating hypertriglyceridemia, cardiovascular disease, metabolic syndrome, Type2 diabetes, obesity, cancer, renal anemia, Alzheimer's disease; and for modulating activity of peroxisome proliferator-activated receptors (PPARs).

### BACKGROUND OF THE INVENTION

Coronary heart disease (CHD) continues to be a leading cause of impaired quality of life and mortality around the world due to a huge rise in obesity, diabetes, and insufficient exercise. Cardiovascular disease is characterized by clogged arteries and reduced blood supply and nutrients to the heart muscle caused by lipid deposition inside the arterial wall. Hyperlipidemia or hyperlipoproteinemia (from lipid-protein complexes) may be caused by genetic factors, obesity or metabolic disorders. Low density lipoproteins (LDL) and very low density lipoproteins (VLDL) are rich in cholesterol and triglycerides, and make up the bulk of the plaque in the arterial wall. LDL-C particles, referred to as the “bad cholesterol”, are produced in the liver from dietary cholesterol or from liver-synthesized cholesterol. Then there is the “good cholesterol”. HDL-C, the high density lipoproteins. HDL-C particles are responsible for a cleansing mechanism called ‘reverse cholesterol transport’ whereby the cholesterol is transported from extra hepatic tissues to the liver for catabolic destruction and excretion. High levels of LDL-C are effectively treated with HMG-CO-A reductase inhibitors (statins), which inhibit the de novo synthesis of cholesterol in the liver. Triglycerides (esters of fatty acids and glycerol) originate from diet, cooking oils, butter and dairy products. It is widely accepted that low levels of HDL-C and high levels of triglycerides in plasma are important risk factors contributing to CHD (U.S. Pat. No. 7,345,190; NCEP Panel. Circulation 1994, 89, 1329). Statins have little impact on reducing elevated plasma levels of triglycerides, and raise HDL only at high doses, (“Effect of statins on HDL-C: a complex process unrelated to changes in LDL-C”; Philip J. Barter, et al., J. Lipid Res., June 2010, 51(6), 1546-1553).

In humans, fibrates, such as clofibrate, bezafibrate, fenofibrate, etofibrate, gemfibrozil, and G-10-2331, have been successfully used to treat hypertriglyceridemia. Fibrates activate peroxisome proliferator activated receptor (PPAR) α, increasing the activity of lipoprotein lipase, which causes a decrease in triglyceride levels. LDL changes from small, dense morphology to large, buoyant particles that are more rapidly cleared by liver. PPARα activation also increases HDL production (Elena Citkowitz et al., “Polygenic Hypercholesterolemia Treatment and Management”, Medscape Reference, Jan. 12, 2012). Oxa-substituted α,ω-alkanedicarboxylic fibric acids raise serum HDL levels significantly, e.g., CI-1027 which has been in clinical trials (Om P. Goel, U.S. Pat. No. 7,345,190; C. L. Bisgaier, et al., U.S. Pat. No. 5,756,544; J. Bar-Tana, et al., U.S. Pat. No. 4,689,344; U.S. Pat. No. 4,711,896).

Omega-3 oils or omega-3 fatty acids are naturally occurring, straight-chain (16-24 carbons) fatty carboxylic acids (PUFAs), essential for normal metabolism in humans and other animals. Since the omega-3 fatty acids are not synthesized by the human body, they are recommended to be taken as dietary supplements in 1-4 grams daily for cardiovascular health benefits, preventing strokes, and reducing blood pressure. (Delgado-Lista, J., et al., “Long Chain Omega-3 Fatty Acids and Cardiovascular Disease: A Systematic Review.” The British Journal of Nutrition, June 2012, 107 Suppl 2, S201-13).

Omega-3 fatty acids have 3-6 conjugated carbon-carbon double bonds and are so named as the first carbon with unsaturation is 3^{rd} carbon from the distal carboxylic acid carbon. All double bonds are in the cis configuration. Among the omega-3 fatty acids eicosapentanenoic acid (EPA, 20 carbons, 5 conjugated double bonds), docohexaenoic acid (DHA, 22 carbons, 6 conjugated double bonds) and α-linolenic acid (ALA, 18 carbons, 3 conjugated double bonds) are the most studied PUFAs pharmacologically. Pharmaceutically effective mixtures of ethyl esters of eicosapentaenoic acid and docosahexaenoic acid are prescribed to treat hypertriglyceridemia. For example, the drug Lovaza™ (developed by Reliant Pharmaceuticals and marketed by GlaxoSmithKline (GSK)) is approved by the US FDA to lower very high triglyceride levels ≧2500 mg/dl. One 1 g capsule contains approximately 465 mg of EPA and 375 mg of DHA. In July 2012, the US FDA approved Amarin's Vascepa™ (icosapent ethyl, EPA ethyl ester) for treating severe hypertriglyceridemia (U.S. Pat. No. 8,188,146). In May, 2014, the US FDA approved Astra-Zeneca's Epanova® to reduce high triglycerides. Epanova contains fatty acids EPA and DHA in their free acid form at a concentration of 50-60% w/w of EPA and 15-25% w/w of DHA.

The mechanisms by which omega-3 fatty acids lower circulating triglycerides are being actively studied. One theory is that the omega-3 fatty acids inhibit the formation of VLDL particles in the liver, which in turn lowers the level of circulating triglycerides. Eicosopentaneoic acid (20:5 EPA), increases fatty acid and glucose uptake and glucose oxidation in cultured human skeletal muscle cells (Aas, Vigdis et al., J. of Lipid Res., 2006, 47, 366-374. It is possible that they act through similar cellular pathways of lipid and lipoprotein metabolism, such as induction of the beta-oxidation pathway similar to fibric acids, such as benzfibrate, fenofibrate and gemfibrozil. The fibrates are peroxisome proliferator-activator receptor alpha (PPARα) agonists, the omega-3 acids DHA and EPA are also mild peroxisome proliferator-activator receptor gamma (PPARγ) activators. Both receptors have a distinct tissue expression; PPARα is expressed at high levels in the liver, whereas PPARγ is expressed in many tissues, with the highest concentrations in adipose and skeletal muscle cells (A. Banga, et al., “Adiponectin translation is increased by the PPARgamma agonists pioglitazone and omega-3 fatty acids”, Am J Physiol Endocrinol. Metab. March 2009, 296(3), 13-14.

Omega-3 polyunsaturated fatty acids (PUFAs) and their metabolites are natural ligands for peroxisome proliferator receptor activator gamma (PPARγ) and, due to the effects of PPARγ on cell proliferation, survival and differentiation, are potential anticancer agents. (Edwards I. J., et al., “Omega-3 Fatty Acids and PPARgamma in Cancer”, 2008, PPAR Res. 358052).

Clearly, finding a compound that would improve fibric acid type derivatives for these purposes would aid in better control of these diseases.

### BRIEF SUMMARY OF THE INVENTION

Omega-3 acids offer an unexplored and unusual structural motif of long aliphatic carbon straight-chains rich with 4-6 conjugated, all cis double bonds of 8-12 π electrons, which is in contrast to a substituted benzene or phenoxy ring (6-8 π electrons) as found in the clinically used present fibrates.

Mono- and poly-unsaturated fatty acids, including the omega-3 acids, have been shown to interact with, and in some cases activate the transcriptional activity of, PPARγ (Xu H E, et al. “Molecular recognition of fatty acids by peroxisome proliferator-activated receptors”, Mol. Cell [Internet]. 1999 Mar. 17, 3(3), 397-403; Kliewer S A, et al., “Fatty acids and eicosanoids regulate gene expression through direct interactions with peroxisome proliferator-activated receptors alpha and gamma”, Proc. Natl. Acad. Sci. USA [Internet], 1997 Apr. 17, 94(9), 4318-23). Because the omega-3 acids are already known to be mild PPARγ and PPARα ligands, and because they have additional non-PPAR-mediated health benefits, the present invention aims to integrate the structural features of some of the commonly prescribed fibrates into the omega-3 acids, referred to herein as Omefibrates, to increase their triglyceride lowering activity, and cardio protective benefits. While not wishing to be bound by theory, the resulting compounds are expected to have “souped-up” PPARs activity, and/or other unique biological properties from combining these PPAR activities. Surprisingly, these compounds having both PUFA-like moieties and a fibrate like functionality have not been synthesized or reported previously.

These modified PUFA derivatives of the present invention are formed by reduction of the carboxylic acid of the omega-3s to an end methylene moiety with a leaving group X, such as a halide, mesylate or tosylate which is displaced by an isobutyrate anion. The following structures depict these present compounds of Formula (I):

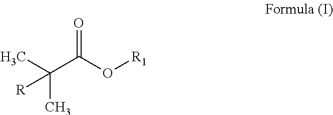

wherein: R₁ is —H, —C₂H₅, —C₃H₇, —CH(CH₃)₂, —C(CH₁)₃, or —C(C₂H₅)(CH₃)₂ or when R₁ is —H and when it is converted to its metformin salt, then R₁ is a metformin cation of the formula

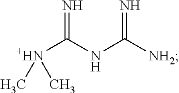

R is joined from the methylene moiety formed by reduction of the carboxylic acid of one of the following polyunsaturated fatty acids (PUFAs):
cis,cis,cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA); cis,cis,cis,cis,cis-5,8,11,14,17-eicosapentanenoic acid (EPA), cis,cis,cis,cis,cis-6,9,12,15,18-heneicosapentaenoic acid (HPA), cis,cis,cis,cis,cis-7,10,13,16,19-docosapentaenoic acid (DPA), cis,cis,cis,cis,cis,cis-4,7,10,13,16,19-docosahexaenoic acid (DHA), cis,cis,cis,cis,cis-9,12,15,18,21-tetracosapentaeonic acid (TPA) or cis,cis,cis,cis,cis,cis-6,9,12,15,18,21-tetracosahexaeonic acid (THA).

The link between diabetes, Type2 (T2D) and dyslipidemia, and resulting coronary heart disease is unequivocal. Dyslipidemia affects 50% of patients with T2D, is characterized by high triglyceride levels, high LDL and low HDL particles (K. Vijayaraghavan, “Treatment of dyslipidemia in patients with Type2 diabetes”, 2010, Lipid Health Dis. 9, 144). These conditions are among the characteristics of what is known as metabolic syndrome. Metabolic syndrome is a disorder of energy utilization and storage, diagnosed by a co-occurrence of three out of five of the medical conditions: abdominal (central) obesity, elevated blood pressure, elevated fasting plasma glucose, high serum triglycerides, and low high-density cholesterol (HDL) levels. Metabolic syndrome increases the risk of developing cardiovascular disease, particularly heart failure, and diabetes (WIKI).

Another purpose of the present invention is to synthesize and study compounds of Formula (II) made from the fibric acids of Formula (I) by a salt forming reaction with metformin, which is widely prescribed to treat Type2 diabetes. Such compounds have the following formula:

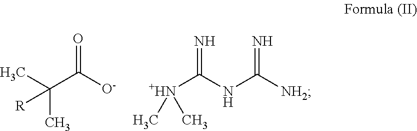

wherein: R is defined as in Formula (I).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph in color showing the results from testing of 3 compounds of the present invention (JIVA-0018 green dots. JIVA-0019 pink dots; JIVA-0020 orange dots) compared to 2 standards (bezafibrate red dots; GW7647 black dots) in a PPARα Agonist Assay where the normalized luciferase activity is plotted vs. the log of the concentration (M) of the tested compound. JIVA-0018 and bezafibrate are at the base line.

FIG. 2 is a graph in color showing the results from testing of 3 compounds of the present invention (JIVA-0018 green dots, JIVA-0019 pink dots; JIVA-0020 orange dots) compared to 1 standard (GW7647 red dots) in a PPARδ Agonist Assay where the normalized luciferase activity is plotted vs. the log of the concentration (M) of the tested compound. JIVA-0018 and JIVA-0019 are at the base line.

FIG. 3 is a graph in color showing the results from testing of 4 compounds of the present invention (JIVA-0013 blue dots. JIVA-0018 green dots, JIVA-0019 pink dots; JIVA-0020 orange dots) of the tested compound compared to 1 standard (bezafibrate red dots) in a PPARγ Agonist Assay where the normalized luciferase activity is plotted vs. the log of the concentration (M) of the tested compound.

### DETAILED DESCRIPTION OF THE INVENTION

It is understood that the terminology used herein is for the purpose of describing particular embodiments only and is not intended to be limiting. As used in this specification, the singular forms “a”, “an”, and “the” include plural referents unless the content clearly indicates otherwise. The following terms in the Glossary as used in this application are to be defined as stated below and for these terms, the singular includes the plural.

Various headings are present to aid the reader, but are not the exclusive location of all aspects of that referenced subject matter and are not to be construed as limiting the location of such discussion.

Also, certain US patents and PCT published applications have been incorporated by reference. However, the text of such patents is only incorporated by reference to the extent that no conflict exists between such text and other statements set forth herein. In the event of such conflict, then any such conflicting text in such incorporated by reference US patent or PCT application is specifically not so incorporated in this patent.

### Glossary

ALA means α-linolenic acid or cis,cis,cis-9,12,15-octadecatrienoic acid, having 18 carbons, 3 cis double bonds, that is modified by reduction of the carboxylic acid to a methylene moiety to be R of Formula (I), (11Z,14Z,17Z)-eicosa-11,14,17-trien-2-yl)-2,2-dimethyleicosanoic acid, and its ethyl, n-propyl, isopropyl, t-butyl, and dimethylethyl esters, as shown by the formula below:

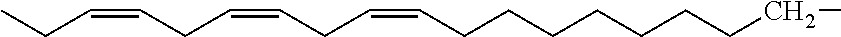 [SMILES: CC/C=C\C/C=C\C/C=C\CCCCCCCCC]

DHA means cis,cis,cis,cis,cis,cis-4,7,10,13,16,19-docosahexaenoic acid or docosahexaenoic acid, having 22 carbons, 6 cis double bonds, that is modified by reduction of the carboxylic acid to a methylene moiety to be R of Formula (I), (6Z,9Z, 12Z,15Z,18Z,21Z)tetracosa-69,12,15,18,21-hexaen-2-yl)-2,2-dimethyl-tetracosanoic acid, and its ethyl, n-propyl, isopropyl, t-butyl, and dimethylethyl esters, as shown by the formula below:

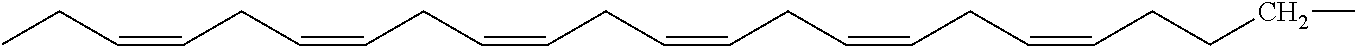 [SMILES: CC/C=C\C/C=C\C/C=C\C/C=C\C/C=C\C/C=C\CCCC]

DPA means cis,cis,cis,cis,cis-7,10,13,16,19-docosapentaenoic acid or docosapentaenoic acid, having 22 carbons, 5 cis double bonds, that is modified by reduction of the carboxylic acid to a methylene moiety to be R of Formula (I), (9Z,12Z, 15Z,18Z,21Z)tetracosa-9,12,15,18,21-pentaen-2-yl)-2,2-dimethyltetracosanoic acid, and its ethyl, n-propyl, isopropyl, t-butyl, and dimethylethyl esters, as shown by the formula below:

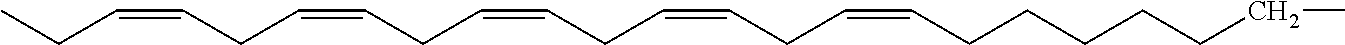 [SMILES: CC/C=C\C/C=C\C/C=C\C/C=C\C/C=C\CCCCCCC]

EPA means cis,cis,cis,cis,cis-5,8,11,14,17-eicosapentanenoic acid or eicosapentanenoic acid, having 20 carbons, 5 cis double bonds, that is modified by reduction of the carboxylic acid to a methylene moiety to be R of Formula (I), (7Z,10Z,13Z,16Z,19Z)-docosa-7,10,13,16,19-pentaen-2-yl)-2,2-dimethyldocosanoic acid, and its ethyl, n-propyl, isopropyl, t-butyl, and dimethylethyl esters, as shown by the formula below:

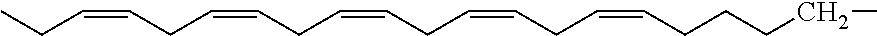 [SMILES: CC/C=C\C/C=C\C/C=C\C/C=C\C/C=C\CCCCC]

ETA means cis,cis,cis,cis-8,11,14,17-eicosatetranoic acid or eicosatetraenoic acid, having 20 carbons, 4 cis double bonds, that is modified by reduction of the carboxylic acid to a methylene moiety to be R of Formula (I), (10Z,13Z,16Z,19Z)-docosa-10,13,16,19-tetraen-2-yl)-2,2-dimethyldocosanoic acid, and its ethyl, n-propyl, isopropyl, t-butyl, and dimethylethyl esters, as shown by the formula below:

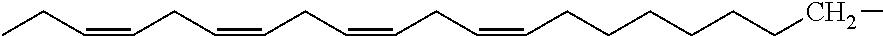 [SMILES: CC/C=C\C/C=C\C/C=C\C/C=C\CCCCCCCC]

ETE means cis,cis,cis-11,14,17-eicosatrienoic acid or eicosatrienoic acid, having 20 carbons, 3 cis double bonds, that is modified by reduction of the carboxylic acid to a methylene moiety to be R of Formula (I), (13Z,16Z,19Z)-docosa-13,16,19-triene-2-yl)-2,2-dimethyldocosanoic acid, and its ethyl, n-propyl, isopropyl, t-butyl, and dimethylethyl esters, as shown by the formula below:

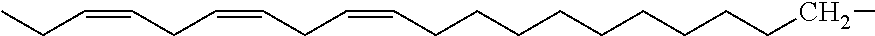 [SMILES: CC/C=C\C/C=C\C/C=C\CCCCCCCCCCC]

HPA means cis,cis,cis,cis,cis-6,9,12,15,18-heneicosapentaenoic acid or heneicosapentaenoic acid, having 21 carbons, 5 cis double bonds, that is modified by reduction of the carboxylic acid to a methylene moiety to be R of Formula (I), (8Z 11Z,14Z, 17Z,20Z)-tricosa-8,11,14,17,20-pentaen-2-yl)-2,2-dimethyltricosanoic acid, and its ethyl, n-propyl, isopropyl, t-butyl, and dimethylethyl esters, as shown by the formula below:

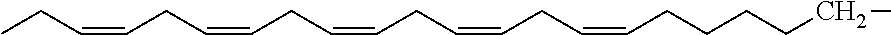 [SMILES: CC/C=C\C/C=C\C/C=C\C/C=C\C/C=C\CCCCCC]

HTA means cis,cis,cis-7,10,13-hexadecatrienoic acid, having 16 carbons, 3 cis double bonds, that is modified by reduction of the carboxylic acid to a methylene moiety to be R of Formula (I), (9Z,12 Z, 15Z)-octadeca-9,12,15-trien-2-yl)-2,2-dimethyloctadecanoic acid, and its ethyl, n-propyl, isopropyl, t-butyl, and dimethylethyl esters, as shown by the formula below:

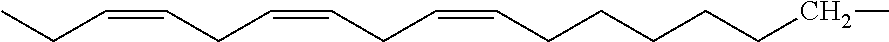 [SMILES: CC/C=C\C/C=C\C/C=C\CCCCCCC]

SDA means cis,cis,cis,cis-6,9,12,15-octadecatetraenoic acid or stearidonic acid, having 18 carbons. 4 cis double bonds, that is modified by reduction of the carboxylic acid to a methylene moiety to be R of Formula (I), (8Z,11Z,14Z,17Z)-eicosa-8,11,14,17-tetraen-2-yl)-2,2-dimethyleicosanoic acid, and its ethyl, n-propyl, isopropyl, t-butyl, and dimethylethyl esters, as shown by the formula below:

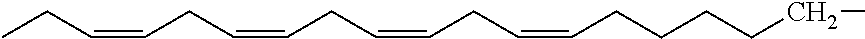 [SMILES: CC/C=C\C/C=C\C/C=C\C/C=C\CCCCCC]

THA means cis, cis, cis, cis,cis,cis-6,9,12,15,18,21-tetracosahexaeonic acid, having 24 carbons, 6 cis double bonds, that is modified by reduction of the carboxylic acid to a methylene moiety to be R of Formula (I), (8Z,11 Z, 14Z, 17Z,20Z,23Z)-hexacosa-8,11,14,17,20,23-hexaen-2-yl)-2,2-dimethylhexacosanoic acid, and its ethyl, n-propyl, isopropyl, t-butyl, and dimethylethyl esters, as shown by the formula below:

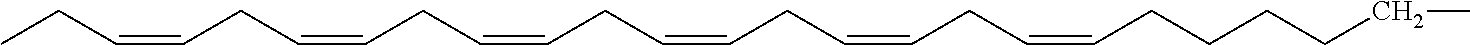 [SMILES: CC/C=C\C/C=C\C/C=C\C/C=C\C/C=C\C/C=C\CCCCCC]

TPA means cis,cis,cis,cis,cis-9,12,15,18,21-tetracosapentaeonic acid, having 24 carbons, 5 cis double bonds, that is modified by reduction of the carboxylic acid to a methylene moiety to be R of Formula (I), (11Z,14Z,17Z,20Z,23Z)-hexacosa-11,14,17,20,23-pentaen-2-yl)-2,2-dimethylhexacosanoic acid, and its ethyl, n-propyl, isopropyl, t-butyl, and dimethylethyl esters, as shown by the formula below:

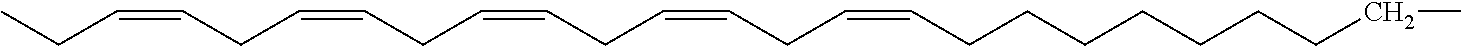 [SMILES: CC/C=C\C/C=C\C/C=C\C/C=C\C/C=C\CCCCCCCCC]

Brine means a saturated solution of sodium chloride in water at room temperature (RT), typically 26% concentration w/v.
DMSO means dimethylsulfoxide
Omega-3 fatty acids means naturally occurring, straight-chain C₁₆-C₂₄ fatty carboxylic acids
PUFA means polyunsaturated fatty acids that are either naturally occurring omega-3 fatty acids or derivatives thereof.
DIBALH means diisobutylaluminumhydride
hr means hour(s)
LDA means lithium diisopropylamide
LAH means lithium aluminumhydride
min. means minute(s)
Omefibrates means fibric acid type derivatives of omega-3 polyunsaturated acids (fatty acids)
Ph means phenyl
RT means room temperature or ambient temperature or about 22 to about 25° C.
rt means retention time in the context of high performance liquid chromatography to determine purity of a compound
THF means tetrahydrofuran
TLC means thin layer chromatography
HRMS means high resolution mass spectroscopy
w/w means weight by weight
w/v means weight per volume

The present invention provides fibrate compounds of Formula (I) that are derived from the above polyunsaturated omega-3 fatty acids (PUFAs) as triglyceride reducing agents of the formula

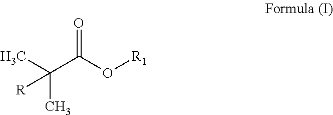

wherein: R₁ is —H, —C₂H₅, —C₃H₇, —CH(CH₃)₂, —C(CH₃)₃ or —C(C₂H₅)(CH₃)₂ or when R₁ is —H and when it is converted to its metformin salt then R₁ is a metformin cation of the formula

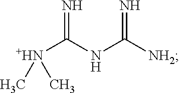

Therefore when the metformin salt is formed from Formula (I) it is shown by Formula (II) and is used to treat metabolic syndrome.

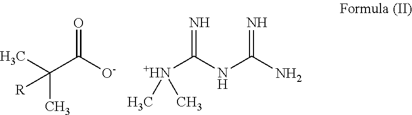

wherein: in both Formula (I) and (II):
R is joined from the methylene moiety formed by reduction of the carboxylic acid of one of the following polyunsaturated fatty acids (PUFAs): cis,cis,cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA); cis,cis,cis,cis,cis-5,8,11,14,17-eicosapentanenoic acid (EPA), cis,cis,cis,cis,cis-6,9,12,15,18-heneicosapentaenoic acid (HPA), cis,cis,cis,cis,cis-7,10,13,16,19-docosapentaenoic acid (DPA), cis,cis,cis,cis,cis,cis-4,7,10,13,16,19-docosahexaenoic acid (DHA), cis,cis,cis,cis,cis-9,12,15,18,21-tetracosapentaeonic acid (TPA) or cis,cis,cis,cis,cis,cis-6,9,12,15,18,21-tetracosahexaeonic acid (THA).

Because the omega-3 acids are already known to be mild PPARγ agonists, the present invention utilizes the formation of compounds by modifying the carboxylic acid of the PUFA and covalently joining an isobutyrate functionality and tests if these compounds have “souped-up” PPARγ activity, and/or other unique biological properties. Such compounds of Formula (I) can be used alone as a pharmaceutically-acceptable formulation, such as a tablet, hard or soft gelatin capsule, or other formulations, or in combination with metformin (Formula II) in treating metabolic syndrome, and also assuring safe cardiovascular health.

### Alzheimer' Disease (AD)

The prevalence and incidence of Alzheimer's disease, and its devastating effects on the lives of patients and care giver families are well known. The health care costs to society are onerous, and will continue to grow with the aging population. Enormous strides have been made in understanding the pathology of the disease which leads to the build-up of amyloid plaques in the brain, which are aggregates of amyloid beta (Aβ) peptides. Fundamental advances have been made in discovering inhibitors of the extra-cellular and intra-cellular neuronal biochemical enzymes such as β-secretage (BACE1) or γ-secretase (GS) to stop the amyloid or intraneuronal τ-tangles build-up; and even reverse these processes through treatment with specific monoclonal antibodies. However, in spite of massive scientific research and investments in reversing the cognitive decline of AD, these have yielded scant benefits. Consensus is emerging that the best approach would be to treat patients before the disease has progressed too far, and even before disease symptoms become apparent. Multi-targeted Alzheimer's drugs, for example dual BACE/acetylcholine esterase inhibition or GSM/PPARγ active agents would offer additional benefits (Harrie J. M. Gisjen, et al., “Secretase Inhibitors and Modulators as a Disease-Modifying Approach Against Alzheimer's Disease”, Annual Reports in Medicinal Chemistry, 2012, 47, 55-69).

The presence of omega-3 fatty acids, especially DHA in the brain is ubiquitous. Clinical studies in 4 year old children support the beneficial effects of docohexaenoic acid (DHA) on cognitive function (NCT 00351624; 2006-2008; sponsored by Martek BioSciences Corporation). It would be an interesting study to follow such treated children over decades regarding the incidence of onset of symptoms of Alzheimer's disease relative to the untreated group. In the meantime, it is worth exploring in a prospective study, if the DHA fibrate, a PPARγ agonist, either alone or in combination with a gamma secretase modulator (GSM) or other prescribed clinical agents, would slow down the decline of cognitive function in early stage AD patients.

This invention will be further clarified by a consideration of the following examples of synthesis of compounds of Formula (I) which are intended to be purely exemplary of the present invention.

The procedures are based on reported literature on the synthesis of fibrates (Om P Goel, U.S. Pat. No. 7,345,190; U.S. Pat. No. 7,770,071).

### Scheme 1: Synthesis Overview

The compounds of the invention were synthesized according to Scheme 1 below in good to excellent yields. The structures were all confirmed by NMR spectra, and HRMS or elemental analysis. Purities were determined by HPLC. All fibrate ethyl esters and fibrate acids are a light yellow or colorless oil. The metformin salts are light yellow to off-white waxes, soluble in chloroform, methylene chloride, DMSO, and partially soluble in diethyl ether.

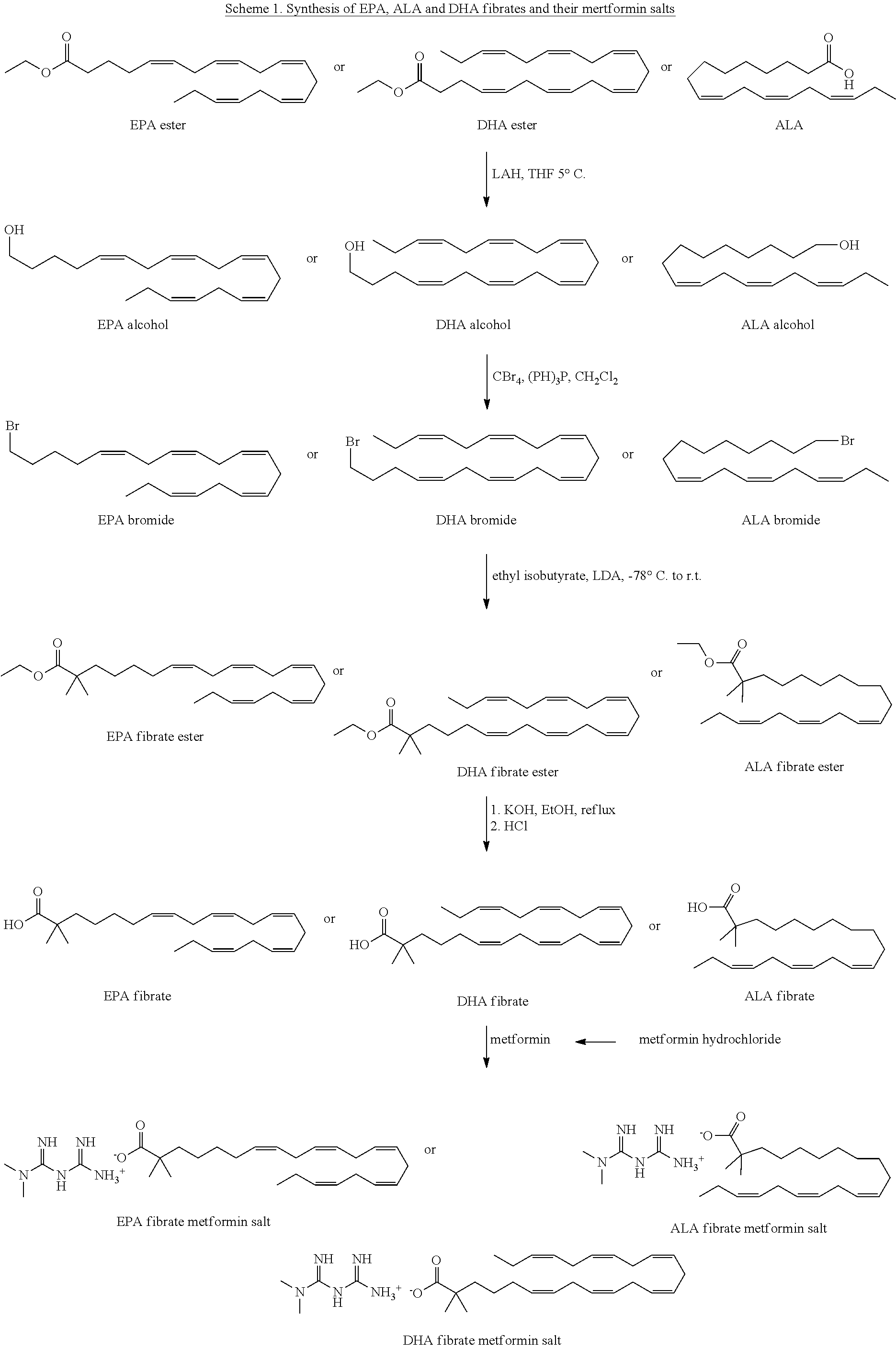

### Example 1: EPA Alcohol, 1

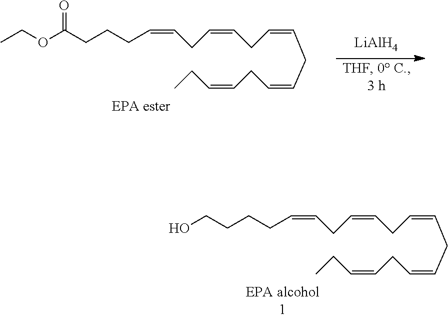 [SMILES: CC/C=C\C/C=C\C/C=C\C/C=C\C/C=C\CCCC(=O)OCC ; "LiAlH4" ; "THF, 0° C.,3 h" ; CC/C=C\C/C=C\C/C=C\C/C=C\C/C=C\CCCCO]

An oven-dried 100 mL round bottomed flask was charged with lithium aluminum hydride (0.76 g, 20 mmol) in anhydrous THF (15 mL). The flask was then cooled to 0° C. with an ice-water bath. To this suspension was added drop-wise a solution of EPA ethyl ester (3.30 g, 10 mmol) in THF (10 mL) via syringe under argon. When the addition was complete, the mixture was stirred for 3 hr at 0° C. The reaction was monitored by TLC. After the reaction was complete, it was quenched at 0° C. by slow drop-wise addition of saturated aqueous solution of sodium sulfate (4 mL). The mixture was stirred for 0.5 hr at RT and then filtered through a Büchner funnel. The residue was rinsed with THF. The filtrate and washings were combined and concentrated under reduced pressure to obtain 2.88 g of EPA alcohol as a yellow oil, yield: 100%, and is characterized by the following spectra:

¹H NMR (300 MHz, CDCl₃/TMS): δ 5.50-5.20 (m, 10H), 3.65 (t, J=6.5 Hz, 2H), 2.92-2.75 (m, 8H), 2.18-2.02 (m, 4H), 1.64-1.54 (m, 2H), 1.64-1.38 (m, 2H), 0.98 (t, J=7.7 Hz, 3H).

¹³C NMR (75 MHz, CDCl₃/TMS): δ 131.8, 129.7, 128.4, 128.2, 128.0, 127.93, 127.86, 127.81, 127.68, 126.8, 62.8, 32.3, 26.9, 25.7, 25.6, 25.5, 20.5, 14.3.

### Example 2: EPA Bromide, 2

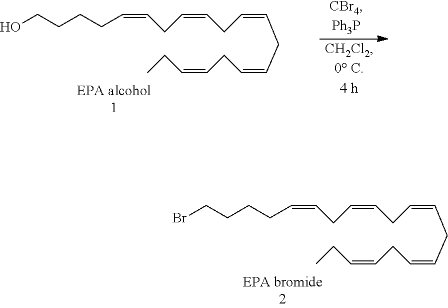

To a solution of EPA alcohol (2.88 g, 10 mmol, prepared as in Example 1) and carbon tetrabromide (3.65 g, 11 mmol) in anhydrous methylene chloride (20 mL) was added triphenylphosphine (2.89 g, 11 mmol) in 4 portions with an interval of 15 min. in between each portion at 0° C. The resulting reaction mixture was stirred at 0° C. The reaction was monitored by TLC. After 4 hr the reaction mixture was concentrated under reduced pressure. Hexanes (30 mL) were added and the mixture was cooled and filtered to remove triphenylphosphine oxide. The filtrate and washings were concentrated under reduced pressure to give crude product as a yellow oil. Purified by silica gel column chromatography (1% ethyl acetate/heptane) provided EPA bromide 2 as colorless oil (3.27 g), yield: 93% and is characterized by the following spectra:

¹H NMR (300 MHz, CDCl₃/TMS): δ 5.45-5.22 (m, 10H), 3.41 (t, J=6.8 Hz, 2H), 2.90-2.70 (m, 8H), 2.15-2.00 (m, 4H), 1.95-1.81 (m, 2H), 1.58-1.42 (m, 2H), 0.98 (t, J=7.7 Hz, 3H).

¹³C NMR (75 MHz, CDCl₃/TMS): δ 131.8, 129.2, 128.4, 128.2, 128.0, 127.9, 127.7, 126.8, 33.6, 32.3, 28.0, 26.3, 25.6, 25.5, 20.5, 14.3.

### Example 3: EPA fibrate ethyl ester, 3 (JIVA-0015)

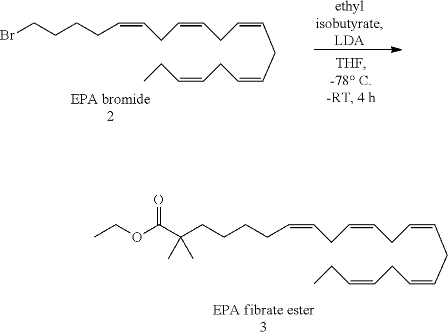 [SMILES: CC/C=C\C/C=C\C/C=C\C/C=C\C/C=C\CCCCBr ; "ethyl isobutyrate,LDA" ; "THF, -78° C.-RT, 4 h" ; CC/C=C\C/C=C\C/C=C\C/C=C\C/C=C\CCCCC(C)(C)C(=O)OCC]

Lithium diisopropylamide solution in THF/Heptane/ethylbenzene (11.0 mL, 2M, 22 mmol) was added drop-wise to a solution of ethyl isobutyrate (2.55 g, 21.9 mmol) in dry THF (15 mL) at −78° C. The resulting light yellow solution was stirred at −78° C. for 1 hr, a solution of EPA bromide (2.57 g, 7.31 mmol, prepared as in Example 2) in anhydrous THF (5 mL) was added drop-wise. Then the reaction mixture was stirred and warmed to RT overnight under argon. The reaction mixture was quenched with ice (10 g), and 1N HCl (5 mL), and diluted with ethyl acetate (30 mL). The phases were separated and the aqueous phase was extracted with ethyl acetate (10 mL×2). The organic layers were combined, washed with water (10 mL), brine (10 mL), dried over MgSO₄, and concentrated under reduced pressure. Purification by silica gel column chromatography (2% EtOAc/heptane) provided the desired EPA fibrate ethyl ester 3 as colorless oil (1.92 g, 68% yield) and is characterized by the following data and spectra:

Chemical Formula: C₂₆H₄₂O₂

Molecular Weight: 386.61

Chromatographic purity (HPLC): 98.7% (rt=7.797 min, 75-100% MeOH/H₂O over 15 min., Luna C18, 5μ, 4.6×250 mm, 1.0 mL/min, 5 μL injection, 40° C., UV detection, 210 nm)

HRMS (DART-ESI-MS): Calculated for C₂₆H₄₆NO₂ (M+NH₄)⁺: 404.3523; found: 404.3534.

¹H NMR (300 MHz, CDCl₃/TMS): δ 5.45-5.22 (m, 10H), 4.11 (q, J=7.0 Hz, 2H), 2.90-2.75 (m, 8H), 2.13-2.00 (m, 4H), 1.55-1.46 (m, 2H), 1.38-1.18 (m, 4H), 1.24 (t, J=7.1 Hz, 3H), 1.15 (s, 6H), 0.98 (t, J=7.7 Hz, 3H).

¹³C NMR (75 MHz, CDCl₃/TMS): δ 177.7, 131.8, 129.9, 128.34, 128.26, 127.98, 127.95, 127.74, 127.68, 127.55, 126.8, 60.1, 42.1, 40.5, 30.1, 27.1, 25.6, 25.5, 25.1, 24.6, 20.5, 14.2.

### Example 4: EPA Fibrate or EPA-Fibric Acid, 4 (JIVA-0018)

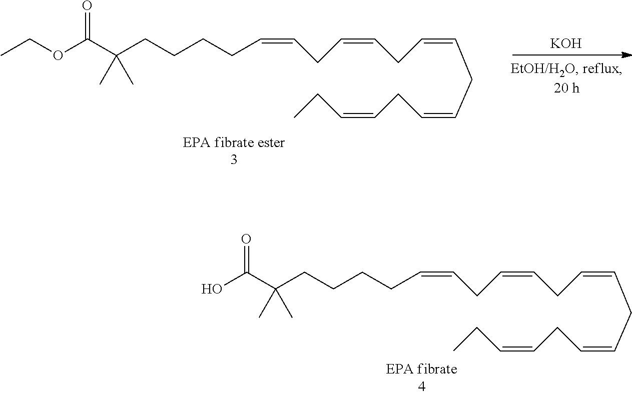 [SMILES: CC/C=C\C/C=C\C/C=C\C/C=C\C/C=C\CCCCC(C)(C)C(=O)OCC ; "KOH" ; "EtOH/H2O, reflux,20 h" ; CC/C=C\C/C=C\C/C=C\C/C=C\C/C=C\CCCCC(C)(C)C(=O)O]

A solution of EPA fibrate ester 3 (1.48 g, 3.83 mmol, prepared as in Example 3) and potassium hydroxide (85%, 0.65 g, 9.85 mmol) in ethanol (10 mL) and water (4 mL) was heated to reflux for 20 hr under argon. The ethanol was evaporated under reduced pressure and the remaining mixture was diluted with water (15 mL). After acidification with aqueous 1N HCl to pH=3, the suspension was extracted with ethyl acetate (3×20 mL). The combined organic phase was washed with brine (10 mL), dried over MgSO₄, and concentrated to give a yellow oil. Purification by silica gel flash chromatography (5% ethyl acetate/heptane) provided desired EPA fibrate 4 (1.30 g, 94% yield) as light yellow oil and is characterized by the following data and spectra:

Chemical Formula: C24H₃₈O₂

Molecular Weight: 358.56

Chromatographic purity (HPLC): 99.6% (rt=13.067 min, 83-100% MeOH/H₂O over 15 min., Luna C18, 5μ, 4.6×250 mm, 1.0 mL/min, 5 μL injection, 40° C., UV detection, 210 nm)

Elemental analysis: Calculated for C₂₄H₃₈O₂: C, 80.39; H, 10.68; found: C, 80.66; H, 10.81.

¹H NMR (300 MHz, CDCl₃/TMS): δ 5.42-5.20 (m, 10H), 2.90-2.72 (m, 8H), 2.13-2.00 (m, 4H), 1.58-1.50 (m, 2H), 1.41-1.22 (m, 4H), 1.19 (s, 6H), 0.97 (t, J=7.5 Hz, 3H).

¹³C NMR (75 MHz, CDCl₃/TMS): δ 184.1, 131.8, 129.9, 128.33, 128.25, 127.97, 127.96, 127.76, 127.69, 127.60, 126.8, 42.0, 40.3, 30.0, 27.0, 25.6, 25.5, 24.9, 24.5, 20.5, 14.2.

### Example 5: EPA Fibrate Metformin Salt, 5 (JIVA-0021)

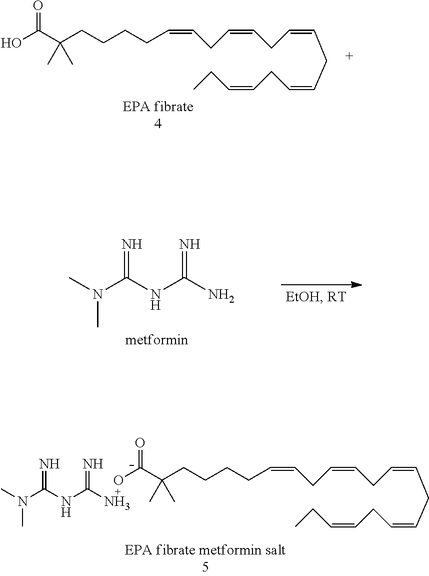

To a solution of starting EPA fibric acid (0.60 g, 1.67 mmol, prepared as in Example 4) in absolute ethanol (3 mL) was added a solution of metformin (0.24 g, 1.84 mmol) in absolute ethanol (2 mL) at RT under argon. The resulting solution was stirred at RT for 2 hr. The ethanol was removed under reduced pressure under 30° C. The residue was dissolved in ether (3 mL) and cooled at −10° C. overnight. Filtration provided desired salt 5 (0.45 g, 55% yield) as yellow wax and is characterized by the following data and spectra:

Chemical Formula: C₂₈H₄₉N₅O₂

Molecular Weight: 487.72

Chromatographic purity (HPLC): 98.9% (rt=14.667 and 19.819 min, 83-100% MeOH/H₂O over 15 min., Alltima C18, 5μ, 4.6×250 mm, 1.0 mL/min, 5 μL injection, 40° C., UV detection, 210 nm)

Elemental analysis: Calculated for C₂₈H₄₉N₅O₂0.85H₂O: C, 66.86; H, 10.16; N; 13.92;

found: C, 65.94; H, 10.24; N; 14.87.

¹H NMR (300 MHz, CDCl₃/TMS): δ 5.80-4.80 (m, 15H), 3.02 (s, 6H), 2.90-2.65 (m, 8H), 2.20-1.95 (m, 4H), 1.50-1.38 (m, 2H), 1.38-1.20 (m, 5H), 1.08 (s, 6H), 0.97 (t, J=7.5 Hz, 3H).

¹³C NMR (75 MHz, CDCl₃/TMS): δ 185.5, 160.4, 158.5, 131.9, 130.5, 128.5, 128.4, 128.1, 127.8, 127.3, 126.9, 43.1, 41.7, 37.6, 30.6, 27.4, 26.3, 25.7, 25.1, 20.6, 14.2.

### Example 6: ALA Alcohol, 6

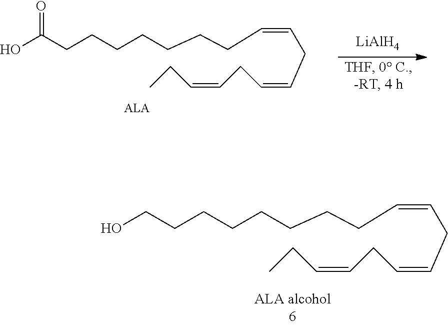 [SMILES: CC/C=C\C/C=C\C/C=C\CCCCCCCC(=O)O ; "LiAlH4" ; "THF, 0° C.,-RT, 4 h" ; CC/C=C\C/C=C\C/C=C\CCCCCCCCO]

An oven-dried 100 mL round bottomed flask was charged with lithium aluminum hydride (2.04 g, 53.87 mmol) in anhydrous THF (20 mL). The mixture was cooled to 0° C. with an ice-water bath. To this suspension was added drop-wise a solution of ALA (5.00 g, 17.96 mmol) in THF (20 mL) via syringe under argon. When the addition was complete, the mixture was allowed to warm to RT and stirred for 4 hr. The reaction mixture was then quenched at 0° C. by slow drop-wise addition of a saturated aqueous solution of sodium sulfate (10 mL). The mixture was then allowed to stir for 0.5 hr at RT and then filtered through a Büchner funnel. The residue was rinsed with THF. The filtrate and washings were combined and concentrated under reduced pressure to obtain 4.75 g of ALA alcohol as colorless oil (100% yield) and is characterized by the following spectra:

¹H NMR (300 MHz, CDCl₃/TMS): δ 5.45-5.25 (m, 6H), 3.63 (t, J=6.6 Hz, 2H), 2.81 (t, J=5.7 Hz, 4H), 2.15-2.00 (m, 4H), 1.62-1.45 (m, 3H), 1.42-1.20 (m, 10H), 0.98 (t, J=7.7 Hz, 3H).

¹³C NMR (75 MHz, CDCl₃/TMS): δ 131.7, 130.1, 128.1, 127.5, 126.9, 62.9, 32.7, 29.6, 29.5, 29.4, 29.2, 27.2, 25.7, 25.6, 25.5, 20.5, 14.3.

### Example 7: ALA Bromide, 7

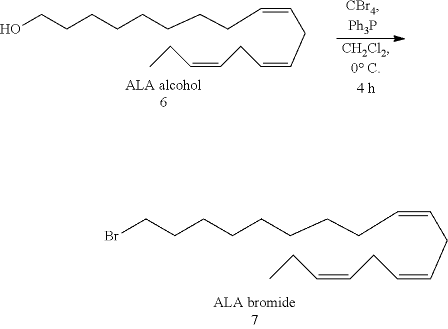

To a solution of ALA alcohol (4.75 g, 17.96 mmol) and carbon tetrabromide (6.55 g, 19.76 mmol) in anhydrous methylene chloride (30 mL) was added triphenylphosphine (5.18 g, 19.76 mmol) in 4 portions with an interval of 15 min. in between each portion at 0° C. The resulting reaction mixture was stirred at 0° C. The reaction was monitored by TLC. After 4 hr, the reaction mixture was concentrated under reduced pressure. Hexanes (50 mL) were added and the mixture was cooled and filtered to remove triphenylphosphine oxide. The filtrate and washings were concentrated under reduced pressure to give crude product as yellow oil. Purified by silica gel column chromatography (1% ethyl acetate/heptane) to provide ALA bromide 7 as colorless oil (5.54 g, 94% yield) and is characterized by the following spectra:

¹H NMR (300 MHz, CDCl₃/TMS): δ5.45-5.25 (m, 6H), 3.40 (t, J=6.9 Hz, 2H), 2.81 (t, J=5.6 Hz, 4H), 2.14-2.00 (m, 4H), 1.95-1.80 (m, 2H), 1.48-1.15 (m, 10H), 0.98 (t, J=7.7 Hz, 3H).

¹³C NMR (75 MHz, CDCl₃/TMS): δ 131.7, 130.0, 128.1, 128.0, 127.5, 126.9, 33.9, 32.8, 29.5, 29.3, 29.1, 28.7, 28.1, 27.2, 25.6, 25.5, 20.5, 14.3.

### Example 8: ALA Fibrate Ethyl Ester, 8 (JIVA-0016)

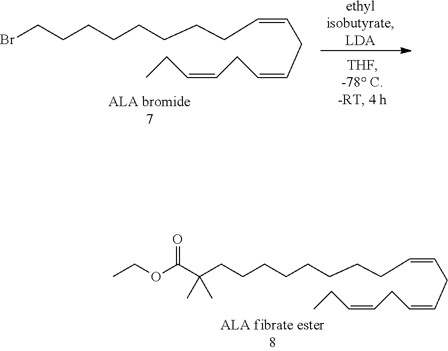 [SMILES: CC/C=C\C/C=C\C/C=C\CCCCCCCCBr ; "ethylisobutyrate,LDA" ; "THF, -78° C.-RT, 4 h" ; CC/C=C\C/C=C\C/C=C\CCCCCCCCC(C)(C)C(=O)OCC]

Lithium diisopropylamide solution in THF/Heptane/ethylbenzene (25.3 mL, 2M, 50.6 mmol) was added drop-wise to a solution of ethyl isobutyrate (5.88 g, 50.59 mmol) in dry THF (25 mL) at −78° C. The resulting light yellow solution was stirred at −78° C. for 1 hr, and a solution of ALA bromide (5.52 g, 16.86 mmol) in anhydrous THF (10 mL) was added drop-wise. Then the reaction mixture stirred and warmed to RT overnight under argon. The reaction mixture was quenched with ice (20 g), and 1N HCl (20 mL), and diluted with ethyl acetate (50 mL). The phases were separated and the aqueous phase was extracted with ethyl acetate (20 mL×2). The organic layers were combined, washed with water (20 mL), brine (20 mL), dried over MgSO₄, and concentrated in vacuo. Purification by silica gel column chromatography (2% ethyl acetate/heptane) provided the desired ALA fibrate ethyl ester 8 as light yellow oil (5.90 g, 96% yield) and is characterized by the following data and spectra:

Chemical Formula: C₂₄H₄₂O₂

Molecular Weight: 362.59

Chromatographic purity (HPLC): 98.9% (rt=8.619 min, 93-100% MeOH/H₂O over 15 min., Luna C18, 5μ, 4.6×250 mm, 1.0 mL/min, 5 μL injection, 40° C., UV detection, 210 nm)

HRMS (DART-ESI-MS): Calculated for C₂₄H₄₆NO₂ (M+NH₄)+: 380.3523; found: 380.3515.

¹H NMR (300 MHz, CDCl₃/TMS): δ 5.55-5.25 (m, 6H), 4.11 (q, J=7.2 Hz, 2H), 2.85-2.75 (m, 4H), 2.13-2.00 (m, 4H), 1.52-1.45 (m, 2H), 1.40-1.05 (m, 15H), 1.15 (s, 6H), 0.98 (t, J=7.7 Hz, 3H).

¹³C NMR (75 MHz, CDCl₃/TMS): δ 177.8, 131.7, 130.2, 128.1, 127.4, 126.9, 60.0, 42.1, 40.7, 30.1, 29.6, 29.4, 29.3, 27.2, 25.6, 25.5, 25.1, 24.9, 20.5, 14.2.

### Example 9: ALA Fibrate, 9 (JIVA-0019)

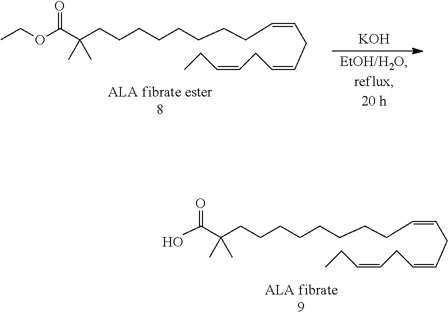 [SMILES: CC/C=C\C/C=C\C/C=C\CCCCCCCCC(C)(C)C(=O)OCC ; "KOH" ; "EtOH/H2O, reflux,20 h" ; CC/C=C\C/C=C\C/C=C\CCCCCCCCC(C)(C)C(=O)O]

A solution of starting ALA fibrate ester 8 (3.50 g, 9.65 mmol, prepared as in Example 8) and potassium hydroxide (85%, 1.82 g, 27.5 mmol) in ethanol (30 mL) and water (12 mL) was heated to reflux for 20 hr under argon. The ethanol was evaporated under reduced pressure and the remaining mixture was diluted with water (45 mL). After acidification with aqueous 1N HCl to pH=3, the formed suspension was extracted with ethyl acetate (3×30 mL). The combined organic phase was washed with brine (20 mL), dried over MgSO₄, and concentrated to give yellow oil. Purification by silica gel flash chromatography (5% ethyl acetate/heptane) provided desired ALA fibrate 9 (3.04 g, 94% yield) as light yellow oil and is characterized by the following data and spectra:

Chemical Formula: C₂₂H₃₈O₂

Molecular Weight: 334.54

Chromatographic purity (HPLC): 96.0% (rt=11.179 min, 97-100% MeOH/H₂O over 8 min., Alltima C18, 5μ, 4.6×250 mm, 1.0 mL/min, 2 μL injection, 40° C., UV detection, 210 nm)

Elemental analysis: Calculated for C₂₂H₃₈O₂: C, 78.99; H, 11.45; found: C, 79.04; H, 11.30.

¹H NMR (300 MHz, CDCl₃/TMS): δ 5.45-5.26 (m, 6H), 2.86-2.76 (m, 4H), 2.12-2.00 (m, 4H), 1.58-1.48 (m, 2H), 1.41-1.10 (m, 13H), 1.19 (s, 6H), 0.98 (t, J=7.5 Hz, 3H).

¹³C NMR (75 MHz, CDCl₃/TMS): δ 184.4, 131.7, 130.2, 128.1, 128.0, 127.4, 126.9, 42.1, 40.5, 30.1, 29.6, 29.5, 29.4, 29.3, 27.2, 25.6, 25.5, 24.9, 24.8, 20.5, 14.3.

### Example 10: ALA Fibrate Metformin Salt, 10 (JIVA-0022)

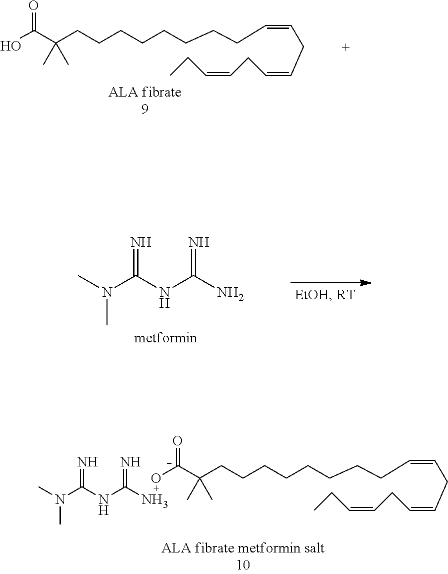

To a solution of ALA fibrate (1.90 g, 5.68 mmol, prepared as in Example 9) in absolute ethanol (5 mL) was added a solution of metformin (0.74 g, 5.68 mmol) in absolute ethanol (3 mL) at RT under argon. The resulting solution was stirred at RT for 2 hr. The ethanol was removed under reduced pressure under 30° C. The residue was dissolved in ether (8 mL) and cooled at −10° C. overnight. Filtration provided desired salt 10 (1.61 g, 61% yield) as off-white wax and is characterized by the following data and spectra:

Chemical Formula: C₂₆H₄₉N₅O₂

Molecular Weight: 463.70

Chromatographic purity (HPLC): 99.8% (rt=13.067 and 23.339 min, 72-100% MeOH/H₂O over 15 min., Alltima C18, 5μ, 4.6×250 mm, 1.0 mL/min, 5 μL injection, 40° C., UV detection, 210 nm)

Elemental analysis: Calculated for C₂₆H₄₉N₅O₂0.5H₂O: C, 66.06; H, 10.66; N; 14.82;

found: C, 66.25; H, 10.49; N; 14.52.

¹H NMR (300 MHz, CDCl₃/TMS): δ 5.80-4.80 (m, 15H), 3.02 (s, 6H), 2.90-2.65 (m, 8H), 2.20-1.95 (m, 4H), 1.50-1.38 (m, 2H), 1.38-1.20 (m, 5H), 1.08 (s, 6H), 0.97 (t, J=7.5 Hz, 3H).

¹³C NMR (75 MHz, CDCl₃/TMS): δ 185.5, 160.4, 158.5, 131.9, 130.5, 128.5, 128.4, 128.1, 127.8, 127.3, 126.9, 43.1, 41.7, 37.6, 30.6, 27.4, 26.3, 25.7, 25.1, 20.6, 14.2.

### Example 11: DHA Alcohol, 11

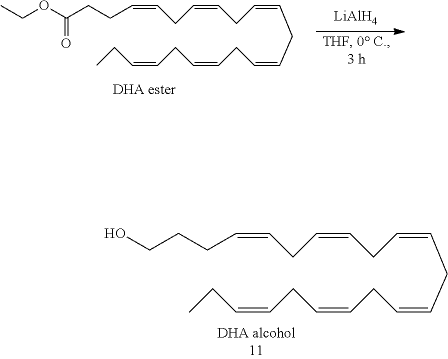 [SMILES: CC/C=C\C/C=C\C/C=C\C/C=C\C/C=C\C/C=C\CCC(=O)OCC ; "LiAlH4" ; "THF, 0° C.,3 h" ; CC/C=C\C/C=C\C/C=C\C/C=C\C/C=C\C/C=C\CCCO]

An oven-dried 100 mL round bottomed flask was charged with lithium aluminum hydride (1.06 g, 28 mmol) in anhydrous THF (15 mL). The flask was cooled to 0° C. with an ice-water bath. To this suspension was added drop-wise a solution of DHA ethyl ester (5.00 g, 14 mmol) in THF (10 mL) via syringe under argon. When the addition was complete, the mixture was allowed to stir for 3 hr at 0° C. The reaction was monitored by TLC. After the reaction was complete, it was quenched at 0° C. by slow drop-wise addition of a saturated aqueous solution of sodium sulfate (5.5 mL). The mixture was then allowed to stir for 0.5 hr at RT and then filtered through a Büchner funnel. The residue was rinsed with THF. The filtrate and washings were combined and concentrated under reduced pressure to obtain 4.41 g of ALA alcohol as light yellow oil (˜100% yield) and is characterized by the following spectra:

¹H NMR (300 MHz, CDCl₃/TMS): δ5.50-5.25 (m, 12H), 3.66 (t, J=6.5 Hz, 2H), 2.95-2.72 (m, 10H), 2.22-2.05 (m, 4H), 1.70-1.55 (m, 2H), 0.98 (t, J=7.7 Hz, 3H).

¹³C NMR (75 MHz, CDCl₃/TMS): δ 131.8, 129.2, 128.4, 128.3, 128.1, 128.0, 127.96, 127.89, 127.7, 126.8, 62.4, 32.4, 25.6, 25.5, 23.6, 20.5, 14.3.

### Example 12: DHA Bromide, 12

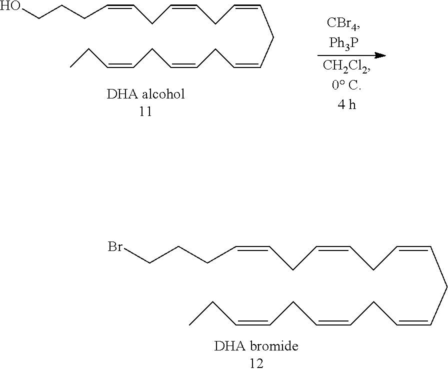

To a solution of DHA alcohol (4.41 g, 14 mmol, prepared as in Example 11) and carbon tetrabromide (5.12 g, 15.4 mmol) in anhydrous methylene chloride (30 mL) was added triphenylphosphine (4.04 g, 15.4 mmol) in 4 portions with an interval of 15 min. in between each portion at 0° C. The resulting reaction mixture was stirred at 0° C. The reaction was monitored by TLC. After 4 hr, the reaction mixture was concentrated under reduced pressure. Hexanes (40 mL) were added, and the mixture was cooled and filtered to remove triphenylphosphine oxide. The filtrate and washings were concentrated under reduced pressure to give crude product as yellow oil. Purified by silica gel column chromatography (1% ethyl acetate/heptane) provide DHA bromide 12 as colorless oil (4.77 g, 90% yield) and is characterized by the following spectra:

¹H NMR (300 MHz, CDCl₃/TMS): 65.48-5.20 (m, 12H), 3.41 (t, J=6.6 Hz, 2H), 2.92-2.70 (m, 10H), 2.30-2.15 (m, 2H), 2.15-2.00 (m, 2H), 2.00-1.85 (m, 2H), 0.97 (t, J=7.5 Hz, 3H).

¹³C NMR (75 MHz, CDCl₃/TMS): δ 131.8, 129.3, 128.4, 128.1, 128.0, 127.92, 127.87, 127.75, 127.66, 126.8, 33.2, 32.4, 25.6, 25.5, 20.5, 14.3.

### Example 13: DHA Fibrate Ethyl Ester, 13 (JIVA-0017)

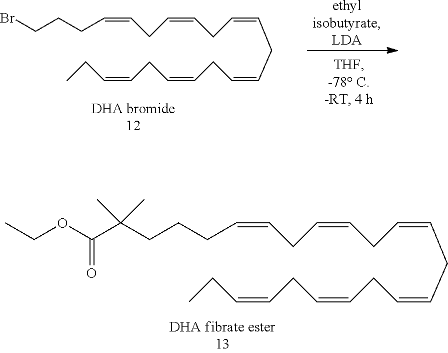 [SMILES: CC/C=C\C/C=C\C/C=C\C/C=C\C/C=C\C/C=C\CCCBr ; "ethylisobutyrate,LDA" ; "THF, -78° C.-RT, 4 h" ; CC/C=C\C/C=C\C/C=C\C/C=C\C/C=C\C/C=C\CCCC(C)(C)C(=O)OCC]

Lithium diisopropylamide solution in THF/Heptane/ethylbenzene (4.0 mL, 2M, 8.0 mmol) was added drop-wise to a solution of ethyl isobutyrate (0.92 g, 7.95 mmol) in dry THF (6 mL) at −78° C. The resulting light yellow solution was stirred at −78° C. for 1 hr. A solution of DHA bromide, (1.00 g, 2.65 mmol) in anhydrous THF (2 mL) was added drop-wise. The reaction mixture stirred and warmed to RT overnight under argon. The reaction mixture was quenched with ice (2 g), and 1N HCl (1 mL), and diluted with ethyl acetate (20 mL). The phases were separated and the aqueous phase was extracted with ethyl acetate (10 mL×2). The organic layers were combined, washed with water (10 mL), brine (10 mL), dried over MgSO₄, and concentrated in vacuo. Purification by silica gel column chromatography (2% ethyl acetate/heptane) provided the desired DHA fibrate ethyl ester 13 as light yellow oil (0.67 g, 61% yield) and is characterized by the following data and spectra:

Chemical Formula: C₂H₄₄O₂

Molecular Weight: 412.65

Chromatographic purity (HPLC): 94.4% (rt=12.683 min, 93-100% MeOH/H₂O over 15 min., Luna C18, 5μ, 4.6×250 mm, 1.0 mL/min, 5 μL injection, 40° C., UV detection, 210 nm)

HRMS (DART-ESI-MS): Calculated for C₂₈H₄8NO₂(M+NH₄)⁺: 430.3680; found: 430.3689.

¹H NMR (300 MHz, CDCl₃/TMS): δ 5.55-5.25 (m, 6H), 4.11 (q, J=7.2 Hz, 2H), 2.85-2.75 (m, 4H), 2.13-2.00 (m, 4H), 1.52-1.45 (m, 2H), 1.40-1.05 (m, 15H), 1.15 (s, 6H), 0.98 (t, J=7.7 Hz, 3H).

¹³C NMR (75 MHz, CDCl₃/TMS): δ 177.8, 131.7, 130.2, 128.1, 127.4, 126.9, 60.0, 42.1, 40.7, 30.1, 29.6, 29.4, 29.3, 27.2, 25.6, 25.5, 25.1, 24.9, 20.5, 14.2.

### Example 14: DHA Fibrate, 14 (JIVA-0020)

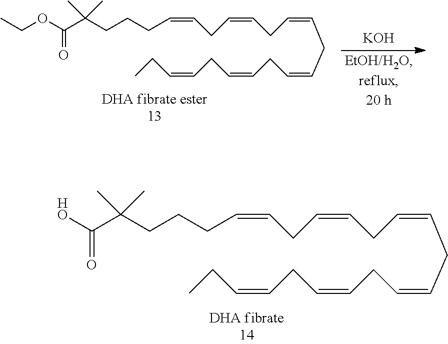

A solution of starting DHA fibrate ester 13 (1.74 g, 4.22 mmol, prepared as in Example 13) and potassium hydroxide (85%, 0.72 g, 10.82 mmol) in ethanol (10 mL) and water (4 mL) was heated to reflux for 20 hr under argon. The ethanol was evaporated under reduced pressure and the remaining mixture was diluted with water (15 mL). After acidification with aqueous 1N HCl to pH=3, the formed suspension was extracted with ethyl acetate (3×20 mL). The combined organic phase was washed with brine (10 mL), dried over MgSO₄, and concentrated to give a yellow oil. Purification by silica gel flash chromatography (5% ethyl acetate/heptane) provided desired DHA fibrate 14 (1.29 g, 80% yield) as a light yellow oil and is characterized by the following data and spectra:

Chemical Formula: C₂₆H₄₀O₂

Molecular Weight: 384.60

Chromatographic purity (HPLC): 90.7% (rt=12.096 min, 93-100% MeOH/H₂O over 15 min., Alltima C18, 5μ, 4.6×250 mm, 1.0 mL/min, 5 μL injection, 40° C., UV detection, 210 nm)

Elemental analysis: Calculated for C₂₂H₃₈O₂: C, 81.2; H, 10.48; found: C, 80.23; H, 10.38.

¹H NMR (300 MHz, CDCl₃/TMS): δ 5.45-5.26 (m, 6H), 2.86-2.76 (m, 4H), 2.12-2.00 (m, 4H), 1.58-1.48 (m, 2H), 1.41-1.10 (m, 13H), 1.19 (s, 6H), 0.98 (t, J=7.5 Hz, 3H).

¹³C NMR (75 MHz, CDCl₃/TMS): δ 184.4, 131.7, 130.2, 128.1, 128.0, 127.4, 126.9, 42.1, 40.5, 30.1, 29.6, 29.5, 29.4, 29.3, 27.2, 25.6, 25.5, 24.9, 24.8, 20.5, 14.3.

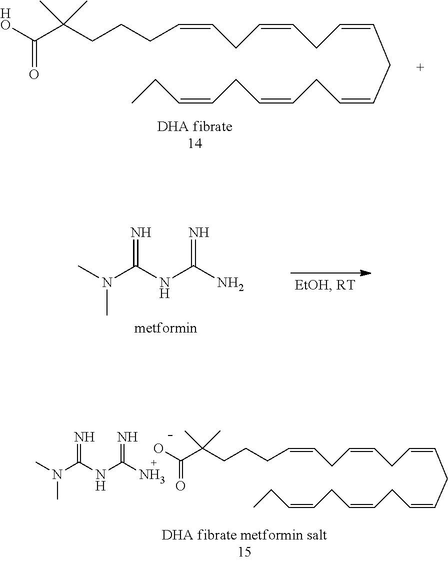

### Example 15: DHA Fibrate Metformin Salt, 15 (JIVA-0023)

To a solution of starting DHA fibrate (0.52 g, 1.35 mmol, prepared as in Example 14) in absolute ethanol (2 mL) was added a solution of metformin (0.18 g, 1.35 mmol) in absolute ethanol (2 mL) at RT under argon. The resulting solution was stirred at RT for 2 hr. The ethanol was removed under reduced pressure under 30° C. The residue was dissolved in ether (2 mL) and cooled at −10° C. overnight. Filtration provided desired salt 15 (0.34 g, 49% yield) as light yellow wax and is characterized by the following data and spectra:

Chemical Formula: C₃₀H₅₁N₅O₂

Molecular Weight: 513.76

Chromatographic purity (HPLC): 94.8% (rt=14.400 and 22.699 min, 72-100% MeOH/H₂O over 15 min., Alltima C18, 5μ, 4.6×250 mm, 1.0 mL/min, 5 μL injection, 40° C., UV detection, 210 nm)

Elemental analysis: Calculated for C₃₀H₅₁N₅O₂0.8H₂O: C, 68.22; H, 10.04; N; 13.26;

found: C, 68.36; H, 10.03; N; 13.13.

¹H NMR (300 MHz, CDCl₃/TMS): δ 5.80-4.80 (m, 15H), 3.02 (s, 6H), 2.90-2.65 (m, 8H), 2.20-1.95 (m, 4H), 1.50-1.38 (m, 2H), 1.38-1.20 (m, 5H), 1.08 (s, 6H), 0.97 (t, J=7.5 Hz, 3H).

¹³C NMR (75 MHz, CDCl₃/TMS): δ 185.5, 160.4, 158.5, 131.9, 130.5, 128.5, 128.4, 128.1, 127.8, 127.3, 126.9, 43.1, 41.7, 37.6, 30.6, 27.4, 26.3, 25.7, 25.1, 20.6, 14.2.

### Utility: In Vitro Biology

The compounds of the present invention are expected to have beneficial effects on metabolic health by activation of the transcription factor PPARα. Although the molecular details are not fully understood, activation of PPARα transcriptional activity increases fatty acid oxidation in multiple tissues, and that this results in a reduction in circulating fatty acids and triglycerides. The activity of JIVA-0018, JIVA-0019 and JIVA-0020 was determined in a PPARα-dependent transcription assay. This assay was carried out in live cells treated with these above compounds, and provides a direct measurement of a compound's ability to activate PPARα. This PPARα activity assay is a standard nuclear receptor ligand activity assay that utilizes the ligand-binding domain of the PPAR receptor fused to a heterologous GAL4 DNA binding domain. The transcriptional read-out is from a GAL4-regulated luciferase reporter. In this assay, compounds that activate the receptor cause an increase in luciferase activity measured in a luminometer. The activity of the invention compounds was compared to two well-characterized PPARα ligands: bezafibrate and GW7647.

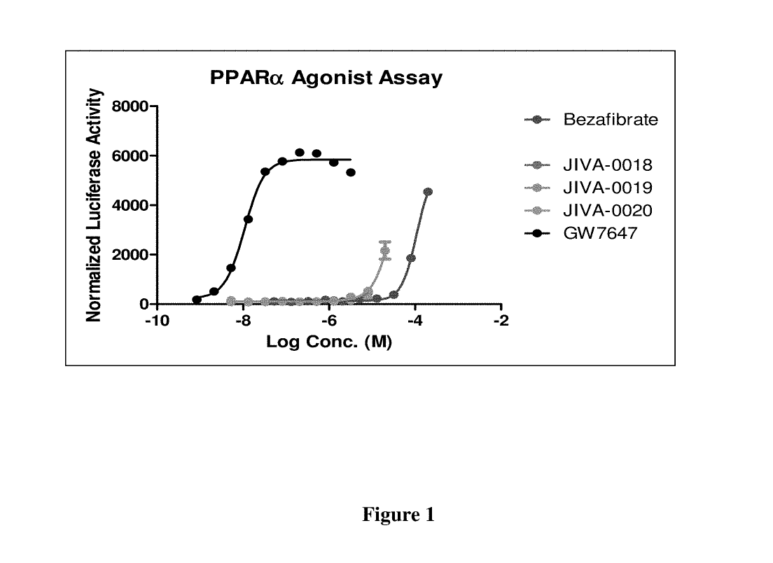

As shown in FIG. 1, while neither the EPA-fibrate, JIVA-0018 nor the DHA-fibrate, JIVA-0020 stimulated PPARα activity, the ALA-fibrate, JIVA-0019, strongly activated PPARα. From these data the potency (EC₅₀) for JIVA-0020 activation of PPARα can be estimated to be somewhat greater than 20 micromolar. This is more potent than benzafibrate, which has an EC₅₀ on PPARα of 100 micromolar in this assay, although it is less potent than GW7647, which has an EC₅₀ of 11 nanomolar.

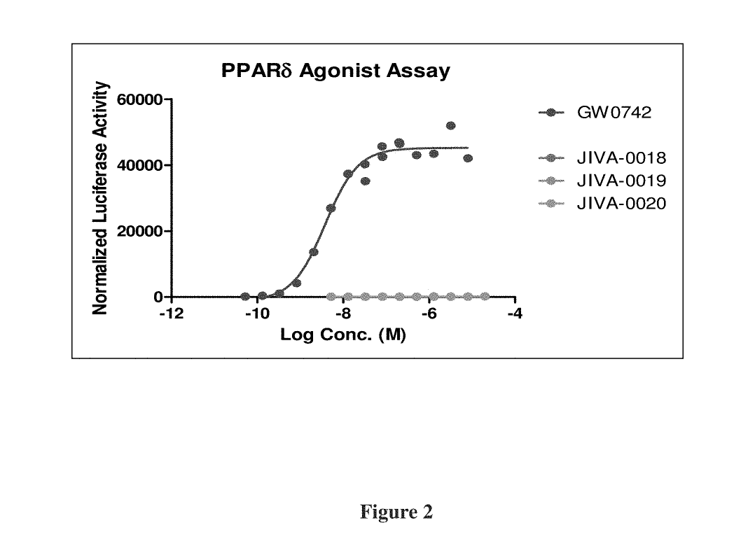

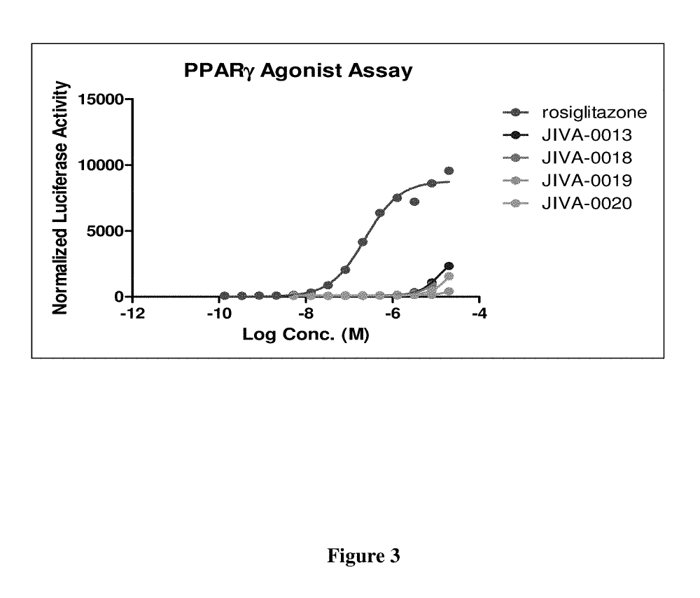

None of the JIVA compounds were active on the related PPAR receptor PPARδ (FIG. 2), and showed only a weak activity on PPARγ (FIG. 3). Thus, it is evident from these data that the ALA-fibrate, JIVA-0019, is a potent and selective activator of PPARα.

Compounds of Formula (I) are preferably used as a pharmaceutically-acceptable formulation such as pharmaceutically-acceptable adjuvants, binders, desiccants, diluents and excipients that are well known for such purpose. Such formulations are in the form of a solution for injection, ampoule, hard or soft gelatin capsule or tablet, or sustained release formulations. These formulations are used to treat persons for Type2 diabetes, obesity, hypertriglyceridemia, cardiovascular diseases, metabolic syndrome, cancer, Alzheimer's disease: and their use for modulating activity of peroxisome proliferator-activated receptors (PPARs).

When the person's the triglycerides levels are in a range of from >100 mg/dl, to >500 mg/dl, then the person needs treatment. An effective amount of the active ingredient in the formulations is from about 0.05 to about 5 g/day administered as 1-4 doses/day.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. A method for treating hypertrilyceridemia, cardiovascular disease, metabolic syndrome, Type2 diabetes, obesity, cancer, renal anemia, or Alzheimer's disease in a arson in need of such treatment by administering an effective amount of a formulation wherein the active ingredient is a compound of Formula (I): 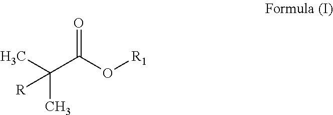
wherein:
R₁ is —H, —C₂H₅, —C₃H₇, —CH(CH₃)₃, —C(CH₃)₃, or —C(C₂H₅)CH₃)₂ or when R₁ is —H and when it is converted to its metformin salt, then R₁ is a metformin cation of the formula 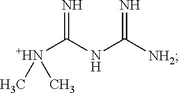
R is joined from the methylene moiety formed by reduction of the carboxylic acid of one of the following polyunsaturated fatty acids (PUFAs): cis,cis,cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA); cis,cis,cis,cis-5,8,11,14,17-eicosapentanenoic acid (EPA), cis,cis,cis,cis-6,12,15,18-heneicosapentaenoic acid (HPA), cis,cis,cis,cis-7,10,13,16,19-docosapentaenoic acid (DPA), cis,cis,cis,cis,cis-4,7,10,13,16,19-docosahexaenoic acid (DHA), cis,cis,cis,cis,cis-9,12,15,18,21-tetracosapentaeonic acid (TPA) or cis,cis,cis,cis,cis,cis-6,9,12,15,18,21-tetracosahexaeonic acid (THA); including its pharmaceutically-acceptable salts, with adjuvant, binders, desiccants, diluents and excipients.

2. The method of claim 1 wherein R₁ is the metformin salt of Formula (I) wherein R₁ is —H; as shown by Formula (II): 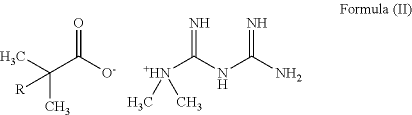
wherein R is defined as in claim 1.

3. The method of claim 1 wherein the compound is ≧90% chemical purity.

4. The method of claim 1 having as its active ingredient one or more compounds of Formula (I) as defined in claim 1.

5. The method of claim 1 wherein the active ingredient is in a formulation in the form of a solution for injection, ampoule, hard or soft gelatin capsule or tablet, or as a sustained release formulation.

6. The method of claim 1 to treat hypertriglyceridemia, Type2 diabetes, or metabolic syndrome in persons needing such treatment by administering to such persons an effective amount of the active ingredient in the formulation.

7. The method of claim 6 to treat metabolic syndrome wherein R₁ in Formula (I) is the metformin cation.

8. The method of claim 6, wherein the triglycerides levels in such persons needing treatment are in a range of from >100 mg/dl, to >500 mg/dl.

9. The method of claim 1 for the treatment of early stages of Alzheimer's disease in persons needing such treatment by administering to such person an effective amount of the active ingredient of the formulation.

10. The method of claim 6, wherein the effective amount is from about 0.05 to about 5 g/day administered as 1-4 doses/day.

11. (canceled)
